(19) United States
(12) Patent Application Publication
(10) Pub. No.: US 2026/0083460 A1
(43) Pub. Date: Mar. 26, 2026

(54) Surgical Drill Bit

(71) Applicant: AmeriDirect Medical, LLC, Palm Beach Gardens, FL (US)

(72) Inventor: Thomas Kennedy, Kentwood, MI (US)

(21) Appl. No.: 18/890,994

(22) Filed: Sep. 20, 2024

(51) Int. Cl. A61B 17/16

(52) U.S. Cl. CPC A61B 17/1617

(57) ABSTRACT

A surgical drill bit includes a shank, a neck connected to the shank, and a fluted region extending from a proximal end connected to the neck to a distal end defining a point angle that is 90 degrees, the fluted region including two flutes separated 180 degrees from each other and defining a cutting portion, wherein the two flutes define a helix having a helix angle and a margin thickness, the helix angle being 25 degrees and the margin thickness being 0.005 inches.

### FIELD

The present disclosure relates generally to instruments designed to be used in surgical procedures, specifically, surgical drill bits.

### BACKGROUND

This section provides background information related to the present disclosure which is not necessarily prior art.

While surgical drill bits are generally known in the art, there is room for improving these devices, particularly by reducing temperature change, decreasing the force required to drill into cortical bone, and decreasing torque experienced while drilling through cortical bone.

### SUMMARY

This section provides a general summary of the disclosure, and is not a comprehensive disclosure of its full scope or all of its features.

One aspect of the disclosure provides a surgical drill bit including a shank, a neck connected to the shank, and a fluted region extending from a proximal end connected to the neck to a distal end defining a point angle that is 90 degrees, the fluted region including two flutes separated 180 degrees from each other and defining a cutting portion, wherein the two flutes define a helix having a helix angle and a margin thickness, the helix angle being 25 degrees and the margin thickness being 0.005 inches.

Implementations of the disclosure may include one or more of the following optional features. In some implementations, the length of the fluted region is 1.4 inches.

The length of the cutting portion may be 1.250 inches.

The length of the surgical drill bit may be 4.8 inches.

The fluted region may include a conical point relief equal to 32 degrees.

The fluted region may include a chisel edge having a chisel angle equal to 122 degrees.

The fluted region may include a tapered web having a web thickness that increases as a diameter of the fluted region increases. The tapered web thickness may include a first web thickness equal to 0.016 inches measured 0.05 inches from the distal end. The tapered web thickness includes a second web thickness equal to 0.04 inches measured 1.15 inches from the distal end.

The body may include a gash having a gash thickness of 0.011 inches. The gash may include a gash angle equal to 80 degrees.

The length of the neck may be equal to between 1.55 inches and 2.15 inches.

A diameter of the fluted region may be 0.098 inches.

Another aspect of the disclosure provides a surgical drill bit including a shank, a neck connected to the shank, and a cannulated fluted region extending from a proximal end connected to the neck to a distal end defining a point angle that is 90 degrees, the fluted region including two flutes separated 180 degrees from each other and defining a cutting portion, wherein the two flutes define a helix having a margin thickness, the margin thickness being 0.005 inches.

Implementations of the disclosure may include one or more of the following optional features. In some implementations, the length of the cannulated fluted region may be 1.4 inches.

The length of the cutting portion may be 1.250 inches.

The length of the surgical drill bit may be 4.8 inches.

The diameter of the fluted region may be 0.098 inches.

The helix may define a helix angle equal to 25 degrees.

The length of the neck may be equal to between 1.55 inches and 2.15 inches.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected configurations and not all possible implementations, and are not intended to limit the scope of the present disclosure.

FIG. 1 is a perspective view of a surgical drill bit in accordance with the present disclosure;

FIGS. 2A-2C are plan views of the surgical drill bit of FIG. 1 shown in different angular orientations;

FIG. 3 is a detailed view of the cutting region or fluted region, of the surgical drill bit of FIG. 1;

FIG. 4 is a detailed view of a portion of the drill point of the surgical drill bit of FIG. 1;

FIG. 5 is a detailed view of a portion of the drill point of the surgical drill bit of FIG. 1;

FIG. 6 is a detailed view of a portion of the drill point of the surgical drill bit of FIG. 1;

FIG. 7 is a plan view of a drill point of the surgical drill bit of FIG. 1; and

FIG. 8 is a graph illustrating an endurance testing comparison for cancellous force between the surgical drill bit and two competitive drill bits.

Corresponding reference numerals indicate corresponding parts throughout the drawings.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

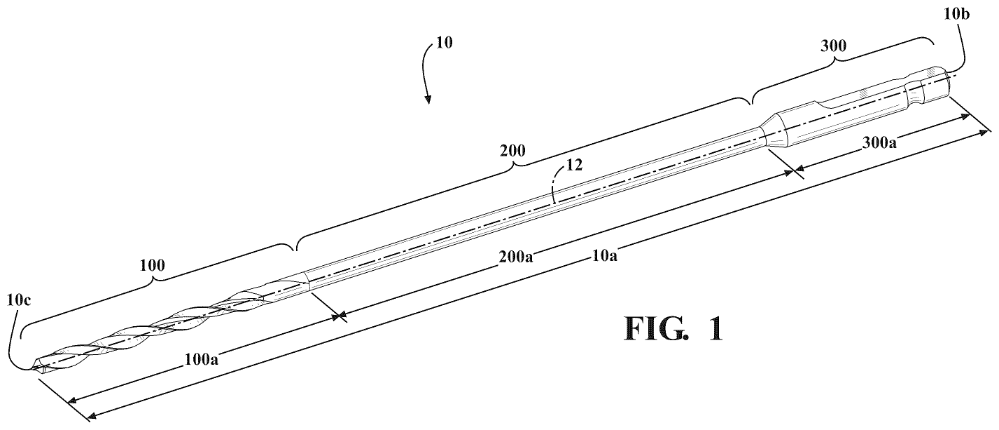

Referring to FIG. 1, a surgical drill bit 10 is generally shown. The surgical drill bit 10 may be designed to be used in a variety of different medical procedures, including, but not limited to, human surgical procedures, veterinarian surgical procedures, etc. The surgical drill bit 10 includes features having extreme values compared to what is available in the market, as well as unique geometry on the outer diameter (OD) relief. As a result of the extreme features and unique geometry, the surgical drill bit 10 performs 2-4 times better compared to known drill bits in the measurements of force and temperature change during drilling on saw bone and 1.4-2 times better torque during drilling on saw bone. The extreme values assigned to each contributing feature of the surgical drill bit 10 are well outside the standard values seen in the industry, such that the surgical drill bit 10 provides a novel and non-obvious design.

Specifically, as will become apparent, the surgical drill bit 10 incorporates the following features, which, when combined with one another, result in the novel design described herein: a particularly high helix angle; an abnormally small web thickness near the drill tip; a high degree of conical cutting edge relief, instead of four facets as is standard in the industry; small drill body margin; and a gash uniquely aligned to the helix and form of the drill flutes to create the surgical drill bit 10 that cuts faster with less effort for the surgeon and less heat to the patient bone. The surgical drill bit 10 has a remarkable ability to perform side-cutting or “milling” once it has entered the bone despite its thinner web thickness which enables varied clinical uses beyond the capabilities of known drill bits. Moreover, the surgical drill bit 10 lasts longer than equivalent comparison drill bits. In addition to abnormal variations in traditional features on a drill bit, the surgical drill bit 10 incorporates a unique geometry on the flute which is a purposeful continuation of the gash onto the OD relief. The surgical drill bit 10 includes a ground relief in the OD relief on the front portion of the drill, which enables more aggressive cutting at initial drilling and allows for more efficient chip evacuation at the drilling site.

With continued reference to FIG. 1, the surgical drill bit 10 comprises a fluted region 100, a neck 200, and a connector or shank 300. The surgical drill bit 10 may be solid as shown in the figures, or, in some implementations, the surgical drill bit 10 may be hollow or cannulated. The surgical drill bit 10 has a length 10a measured from a proximal end 10b to a distal end 10c. In some implementations, the length 10a may be equal to approximately 4.8 inches. The surgical drill bit 10 includes a center axis 12 extending through the center of the surgical drill bit 10 that the surgical drill bit 10 is designed to rotate about during operation. The surgical drill bit 10 may be formed from any suitable material, including surgical grade stainless steel, a titanium alloy, 440A SS per ASTM A276, 50-56 HrC or 440C per ASTM A276, minimum 57 HrC, or any other suitable material.

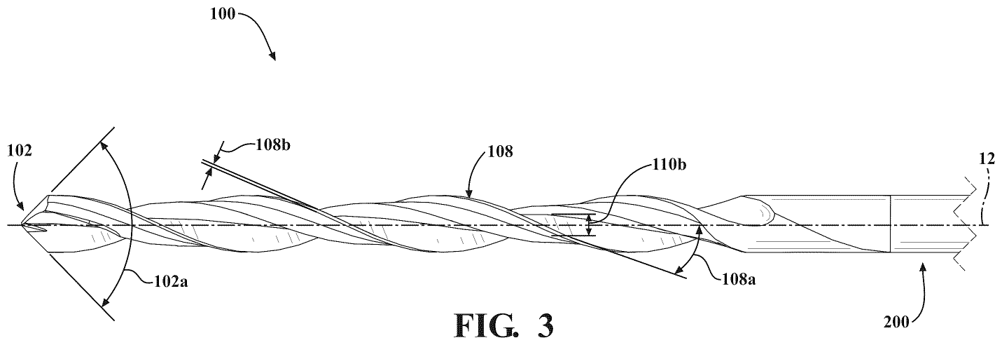

The fluted region 100 includes a length 100a extending from the distal end 10c to the neck 200. In some implementations, the length 100a may be equal to approximately 1.40 inches. The fluted region 100 includes a diameter 100b that, in some implementations, may be equal to between approximately 0.038 inches and 0.177 inches (e.g., 0.0984 inches). The fluted region 100 includes an outer diameter (OD) clearance 100c that, in some implementations, may be equal to between approximately 0.032 and 0.143 inches (e.g., 0.080 inches). The fluted region 100 includes a drill point 102 at the distal end 10c of the drill bit 10. As shown in FIG. 3, the drill point 102 includes a drill point angle 102a, which, in some implementations, may be approximately 90 degrees.

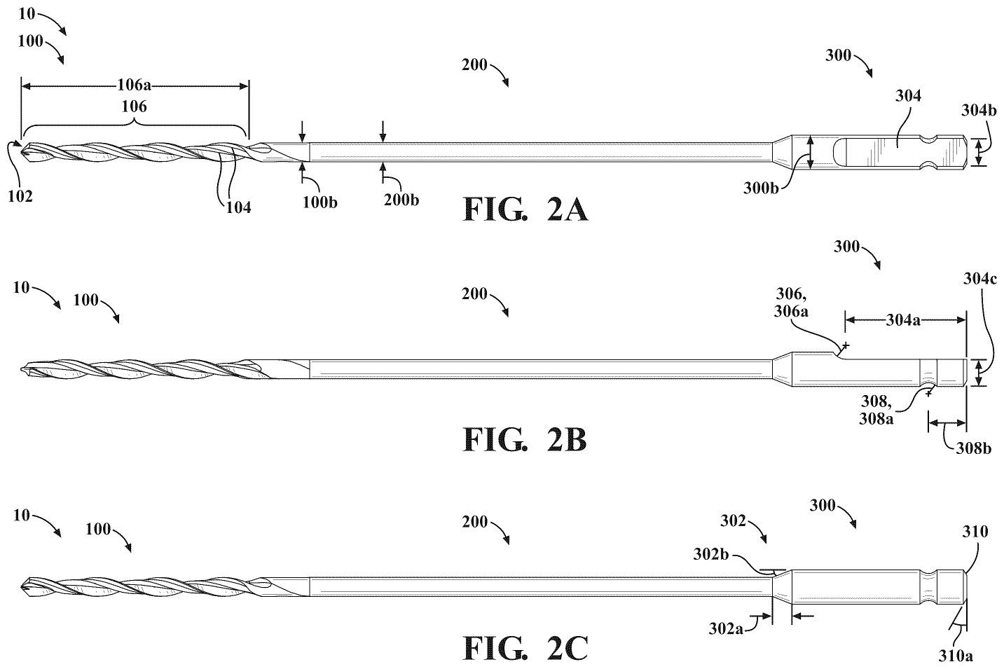

The fluted region 100 includes two flutes 104 that are separated 180 degrees apart from each other and define a cutting portion 106. The cutting portion 106 has a length 106a as shown in FIG. 2A, which, in some implementations, may be equal to approximately 1.250 inches. The flutes 104 define a helix 108 having a helix angle 108a and a margin thickness 108b. In some implementations, the helix angle 108a may be equal to approximately 25 degrees, which reduces the force required to drill. In some implementations, the margin thickness 108b may be equal to between approximately 0.003 inches and 0.007 inches, which significantly reduces the friction between the saw bone and drill resulting in less temperature change and torque during drilling.

Referring to FIGS. 3-7, the fluted region 100 may include a tapered web 110 having a first web thickness 110a measured 0.05 inches from the distal end 10c, and which, in some implementations, may be equal to between approximately 0.010 inches and 0.030 inches (e.g., 0.016 inches) measured at 0.05 inches from the distal end 10c. The web 110 includes a second web thickness 110b measured 1.15 inches from the distal end 10c, and which, in some implementations, may be between approximately 0.025 inches to 0.075 inches (e.g., 0.04 inches). In such a configuration, the web 110 increases in size in a direction extending from the distal end 10c toward the proximal end 10b.

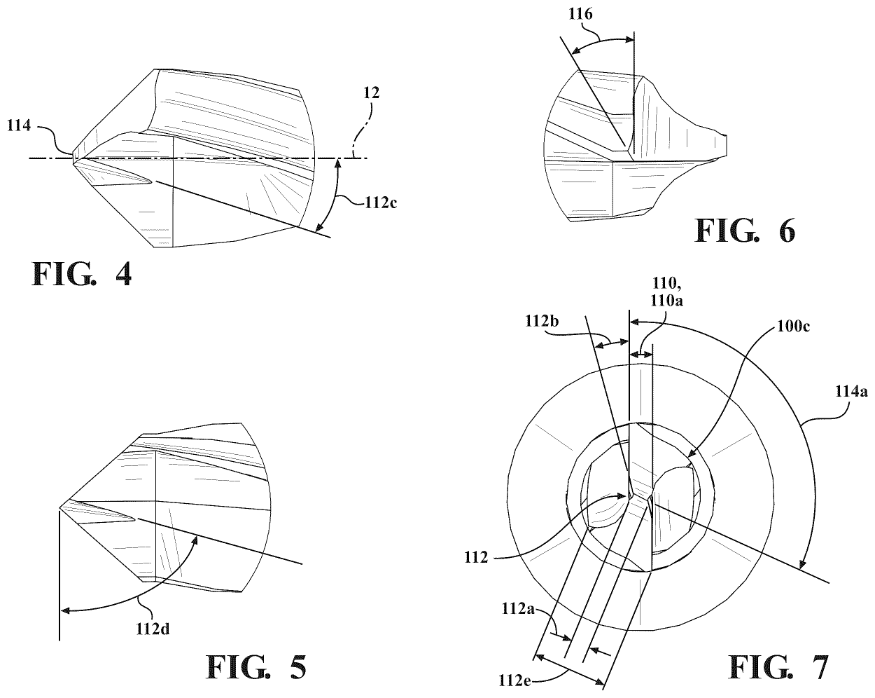

With continued reference to FIGS. 3-7, the fluted region 100 may include a gash 112 extending from a location at or near the drill point 102. As shown in FIG. 7, the gash 112 includes a gash thickness 112a, which, in some implementations, may be equal to between approximately 0.006 inches and 0.020 inches (e.g., 0.011 inches) which enables quick engagement of the drill with cortical bone and reducing drilling force. As shown in FIG. 7, the gash 112 includes a gash rotation angle 112b, which, in some implementations, may be equal to approximately 12 degrees, which enables efficient bone chip formation into the fluted region. As shown in FIG. 4, the gash 112 includes a gash axial rake 112c, which, in some implementations, may be equal to approximately 15 degrees, which directs bone chip formation into the fluted region. As shown in FIG. 5, the gash 112 includes a gash angle normal to gash face 112d, which, in some implementations, may be equal to approximately 80 degrees, which further enables efficient transfer of bone chips into the fluted region. The fluted region 100 may include a land 112e, which, in some implementations, may be equal to between approximately 0.019 and 0.086 inches (e.g., 0.048 inches).

As shown in FIGS. 4 and 7, the fluted region 100 includes a chisel edge 114 having a chisel angle 114a, which, in some implementations, may be equal to approximately 122 degrees, which reduces the force exerted during drilling. As shown in FIG. 6, the fluted region 100 includes a conical point relief 116, which, in some implementations, may be equal to approximately 32 degrees, which reduces the force exerted and torque experienced by the physician as well as significantly reducing the heat generated during drilling.

Referring to FIGS. 1-2C, the drill bit 10 includes the neck 200 extending from the fluted region 100 to the connector 300. The neck 200 may be a generally cylindrical member having a neck length 200a and a neck diameter 200b. In some implementations, the neck length 200a may be equal to approximately 2.15 inches. In some implementations, the neck diameter 200b may be equal to between approximately 0.039 inches and 0.177 inches.

With continued reference to FIGS. 1-2C, the drill bit 10 includes the connector 300 having a connector length 300a extending from the neck 200 to the proximal end 10b and a connector diameter 300b. In some implementations, the connector length 300a may be equal to between approximately 1.25 inches and 1.85 inches. In some implementations, the connector diameter 300b may be equal to approximately 0.1760 inches. The connector 300 includes a taper 302 where the connector 300 meets the neck 200. The taper 302 includes a taper length 302a and a taper angle 302b. In some implementations, the taper angle 302b may be equal to between approximately 30 degrees and 45 degrees.

The connector 300 may include a tang 304 extending to the proximal end 10b. The tang 304 includes a tang length 302a extending from the proximal end. In some implementations, the tang length 302a is equal to approximately 0.750 inches. The tang 304 includes a tang width 304b, which, in some implementations, may be equal to approximately 0.138 inches. The tang 304 may include a tang depth 304c, which, in some implementations, may be equal to approximately 0.139 inches.

The connector 300 includes a groove 308 extending around the circumference of the connector 300. The groove 308 includes a groove radius 308a at a distance 308b from the proximal end 10b. In some implementations, the groove radius 308a may be equal to between 0.045 inches and 0.050 inches, and the distance 308b may be equal to approximately 0.195 inches. The connector 300 may include a chamfer 310 located at or near the proximal end 10b. In some implementations, the chamfer 310 may be equal to approximately 0.040 inches by 45 degrees.

The surgical drill bit 10 includes dimensions that vary based on the diameter 100b, as illustrated in Table 1 below. The dimensions are in inches unless indicated otherwise.

TABLE 1
Drill Dimensions.

| | | | Proximal | | | |
|---|---|---|---|---|---|---|
| | | OD | Web | Distal Web | | Gash |
| | | Clear- | Thick- | Thick- | | Thick- |
| Size | Diameter | ance | ness | ness | Land | ness |
| 1.0 mm | .0394 | .032 | .010 | .025 | .019 | .006 |
| 1.1 mm | .0433 | .035 | .011 | .028 | .021 | .007 |
| 1.5 mm | .0591 | .048 | .013 | .033 | .029 | .008 |
| 1.8 mm | .0709 | .058 | .014 | .035 | .035 | .008 |
| 2.0 mm | .0787 | .064 | .015 | .038 | .038 | .009 |
| 2.2 mm | .0866 | .070 | .015 | .038 | .042 | .010 |
| 2.3 mm | .0906 | .074 | .016 | .040 | .042 | .010 |
| 2.4 mm | .0945 | .077 | .016 | .040 | .046 | .011 |
| 2.5 mm | .0984 | .080 | .016 | .040 | .048 | .011 |
| 2.7 mm | .1063 | .086 | .017 | .042 | .052 | .011 |
| 2.8 mm | .1102 | .090 | .018 | .045 | .054 | .012 |
| 3.2 mm | .1260 | .102 | .021 | .055 | .061 | .013 |
| 3.3 mm | .1299 | .106 | .022 | .05 | .063 | .014 |
| 3.5 mm | .1378 | .112 | .024 | .060 | .067 | .015 |
| 4.0 mm | .1575 | .128 | .027 | .068 | .077 | .017 |
| 4.3 mm | .1693 | .138 | .029 | .073 | .082 | .019 |
| 4.5 mm | .1764 | .143 | .030 | .075 | .086 | .020 |

The surgical drill bit 10 was tested compared to seven competitive and widely used drill bits available on the market. Tables 2-4 below illustrate the results of this testing. The “Comp” column demonstrates the factor improvement of the surgical drill bit 10 (“Subject”) compared to each of the seven other drill bits (“Drill 1-7”).

TABLE 2
Temperature.

| | 0.125 s After Drill Exit | | | | 0.250 s After Drill Exit | | | |
|---|---|---|---|---|---|---|---|---|
| | Abs | | High | Low | Abs | | High | Low |
| Drill | Value | Comp | Std Dev | Std Dev | Value | Comp | Std Dev | Std Dev |
| Subject | 17.8 | — | 22 | 14 | 17 | — | 21 | 13 |
| Drill 1 | 68.5 | 3.86 | 95 | 42 | 65.6 | 3.86 | 92 | 39 |
| Drill 2 | 71.2 | 4.01 | 91 | 52 | 69.2 | 4.08 | 88 | 50 |
| Drill 3 | 73.6 | 4.14 | 124 | 23 | 71.3 | 4.20 | 120 | 22 |
| Drill 4 | 90.7 | 5.11 | 117 | 64 | 86.2 | 5.08 | 111 | 61 |
| Drill 5 | 102.8 | 5.79 | 115 | 90 | 98.4 | 5.80 | 109 | 87 |
| Drill 6 | 105.1 | 5.92 | 128 | 83 | 101.3 | 5.97 | 124 | 79 |
| Drill 7 | 118.5 | 6.67 | 142 | 95 | 113.4 | 6.68 | 137 | 90 |

TABLE 3
Force.

| | Cortical Force | | | | Cancellous Force | | | |
|---|---|---|---|---|---|---|---|---|
| | Abs | | High | Low | Abs | | High | Low |
| Drill | Value | Comp | Std Dev | Std Dev | Value | Comp | Std Dev | Std Dev |
| Subject | 5.4 | — | 6.3 | 4.5 | 0.8 | — | 1.0 | 0.6 |
| Drill 6 | 10.7 | 1.98 | 12.3 | 9.1 | 2.3 | 2.88 | 3.1 | 1.5 |
| Drill 1 | 12.3 | 2.28 | 13.6 | 11.1 | 2.7 | 3.34 | 3.7 | 1.6 |
| Drill 2 | 16.1 | 2.97 | 18.1 | 14.1 | 2.1 | 2.62 | 2.7 | 1.5 |
| Drill 3 | 16.4 | 3.03 | 32.2 | 0.6 | 3.8 | 4.79 | 7.4 | 0.2 |
| Drill 4 | 17.5 | 3.23 | 22.0 | 13.0 | 4.0 | 4.98 | 5.4 | 2.6 |
| Drill 7 | 20.6 | 3.80 | 23.9 | 17.4 | 3.6 | 4.52 | 4.8 | 2.4 |
| Drill 5 | 22.1 | 4.08 | 26.9 | 17.4 | 4.5 | 5.58 | 6.2 | 2.7 |

TABLE 4
Torque.

| | Cortical Torque | | | | Cancellous Torque | | | |
|---|---|---|---|---|---|---|---|---|
| | Abs | | High | Low | Abs | | High | Low |
| Drill | Value | Comp | Std Dev | Std Dev | Value | Comp | Std Dev | Std Dev |
| Subject | 0.39 | — | 0.48 | 0.31 | 0.09 | — | 0.13 | 0.06 |
| Drill 2 | 0.56 | 1.41 | 0.69 | 0.42 | 0.28 | 2.98 | 0.53 | 0.04 |
| Drill 1 | 0.57 | 1.44 | 0.74 | 0.40 | 0.35 | 3.67 | 0.54 | 0.15 |
| Drill 3 | 0.63 | 1.61 | 0.85 | 0.41 | 0.38 | 4.06 | 0.69 | 0.07 |
| Drill 4 | 0.65 | 1.65 | 0.79 | 0.51 | 0.51 | 5.38 | 0.74 | 0.27 |
| Drill 6 | 0.67 | 1.7 | 0.83 | 0.50 | 0.52 | 5.57 | 0.82 | 0.23 |
| Drill 7 | 0.74 | 1.88 | 1.03 | 0.45 | 0.58 | 6.21 | 0.76 | 0.41 |
| Drill 5 | 0.87 | 2.20 | 1.03 | 0.70 | 0.46 | 4.87 | 0.54 | 0.37 |

As shown in Tables 2-4 above, the surgical drill bit 10 (“Subject”) performs a minimum of 3.86 times better than any competitive drill bit for saw bone temperature change observed 0.125 seconds after drill exit, a minimum of 3.86 times better than any competitive drill bit for saw bone temperature change observed 0.250 seconds after drill exit, a minimum of 1.98 times better than any competitive drill bit for force exerted during cortical drilling, a minimum of 2.62 times better than any competitive drill bit for force exerted during cancellous drilling, a minimum of 1.41 times better than any competitive drill bit for torque experienced during cortical drilling, and a minimum of 2.98 times better than any competitive drill bit for torque experienced during cancellous drilling. These significant improvements compared to what exists is attributed to the unique geometry of the surgical drill bit 10 as described herein.

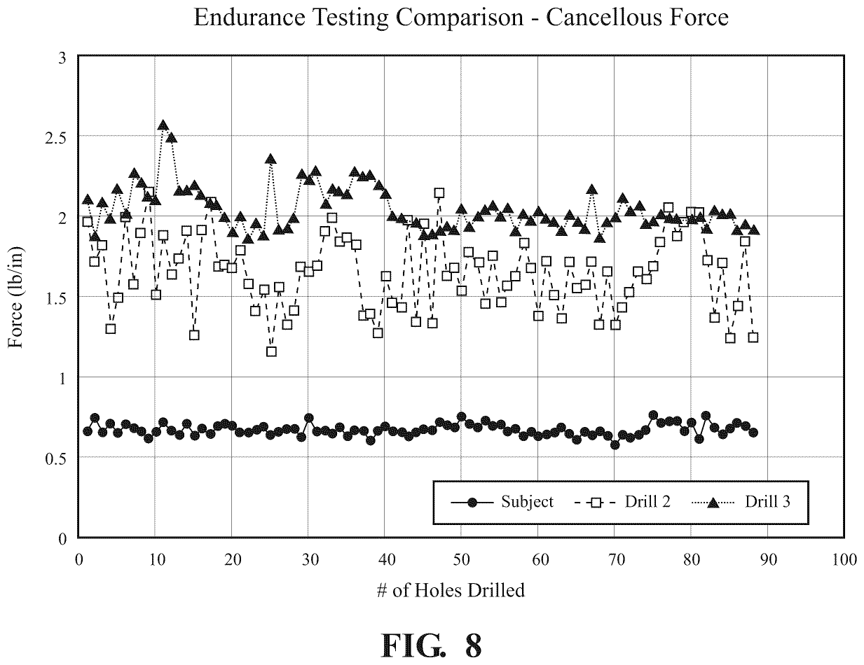

As shown in FIG. 8, the surgical drill bit 10 (“Subject”) performs significantly better than competitive Drill 2 and Drill 3 in endurance testing for cancellous force. These significant improvements compared to what exists is attributed to the unique geometry of the surgical drill bit 10 as described herein.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed above could be termed a second element, component, region, layer or section without departing from the teachings of the example configurations.

The foregoing description has been provided for purposes of illustration and description. It is not intended to be exhaustive or to limit the disclosure. Individual elements or features of a particular configuration are generally not limited to that particular configuration, but, where applicable, are interchangeable and can be used in a selected configuration, even if not specifically shown or described. The same may also be varied in many ways. Such variations are not to be regarded as a departure from the disclosure, and all such modifications are intended to be included within the scope of the disclosure.

What is claimed is:

1. A surgical drill bit comprising:
a shank;
a neck connected to the shank; and
a fluted region extending from a proximal end connected to the neck to a distal end defining a point angle that is 90 degrees, the fluted region including two flutes separated 180 degrees from each other and defining a cutting portion;
wherein the two flutes define a helix having a helix angle and a margin thickness, the helix angle being 25 degrees and the margin thickness being 0.005 inches.

2. The surgical drill bit of claim 1, wherein the length of the fluted region is 1.4 inches.

3. The surgical drill bit of claim 1, wherein the length of the cutting portion is 1.250 inches.

4. The surgical drill bit of claim 1, wherein the length of the surgical drill bit is 4.8 inches.

5. The surgical drill bit of claim 1, wherein the fluted region includes a conical point relief equal to 32 degrees.

6. The surgical drill bit of claim 1, wherein the fluted region includes a chisel edge having a chisel angle equal to 122 degrees.

7. The surgical drill bit of claim 1, wherein the fluted region includes a tapered web having a web thickness that increases as a diameter of the fluted region increases.

8. The surgical drill bit of claim 7, wherein the tapered web thickness includes a first web thickness equal to 0.016 inches measured 0.05 inches from the distal end.

9. The surgical drill bit of claim 8, wherein the tapered web thickness includes a second web thickness equal to 0.04 inches measured 1.15 inches from the distal end.

10. The surgical drill bit of claim 1, wherein the body includes a gash having a gash thickness of 0.011 inches.

11. The surgical drill bit of claim 10, wherein the gash includes a gash angle equal to 80 degrees.

12. The surgical drill bit of claim 1, wherein the length of the neck is equal to between 1.55 inches and 2.15 inches.

13. The surgical drill bit of claim 1, wherein a diameter of the fluted region is 0.098 inches.

14. A surgical drill bit comprising:
a shank;
a neck connected to the shank; and
a cannulated fluted region extending from a proximal end connected to the neck to a distal end defining a point angle that is 90 degrees, the fluted region including two flutes separated 180 degrees from each other and defining a cutting portion;
wherein the two flutes define a helix having a margin thickness, the helix angle being 25 degrees and the margin thickness being 0.005 inches.

15. The surgical drill bit of claim 14, wherein the length of the cannulated fluted region is 1.4 inches.

16. The surgical drill bit of claim 14, wherein the length of the cutting portion is 1.250 inches.

17. The surgical drill bit of claim 14, wherein the length of the surgical drill bit is 4.8 inches.

18. The surgical drill bit of claim 14, wherein the diameter of the fluted region is 0.098 inches.

19. The surgical drill bit of claim 14, wherein the length of the neck is equal to between 1.55 inches and 2.15 inches.

20. The surgical drill bit of claim 14, wherein the fluted region includes a conical point relief equal to 32 degrees.
